(19) United States
(12) Patent Application Publication
(10) Pub. No.: US 2024/0302370 A1
(43) Pub. Date: Sep. 12, 2024

(54) SAMPLE LYSIS REAGENT COMPOSITIONS AND METHODS OF PRODUCTION AND USE THEREOF

(71) Applicant: Siemens Healthcare Diagnostics Inc., Tarrytown, NY (US)

(72) Inventors: Sai Patibandla, Baldwin Place, NY (US); Krishna Singh, New York, NY (US); Geraldine Arrode-Bruses, Chappaqua, NY (US)

(73) Assignee: Siemens Healthcare Diagnostics Inc., Tarrytown, NY (US)

(21) Appl. No.: 18/261,427

(22) PCT Filed: Jan. 14, 2022
(86) PCT No.: PCT/US2022/012514

§ 371 Date: Jul. 13, 2023

Related U.S. Application Data
(60) Provisional application No. 63/200,083, filed on Feb. 12, 2021.

(51) Int. Cl. G01N 33/569

(52) U.S. Cl. CPC G01N 33/56983; G01N 2333/165; G01N 2469/10

(57) ABSTRACT

Methods of utilizing sample lysis reagent compositions in the handling of samples containing viruses, such as (but not limited to) severe acute respiratory syndrome coronavirus 2 (SARS-COV-2), are disclosed. Kits containing the sample lysis reagent compositions are also disclosed.

### CROSS REFERENCE TO RELATED APPLICATIONS/INCORPORATION BY REFERENCE STATEMENT

This application claims benefit under 35 USC § 119(e) of U.S. provisional application Ser. No. 63/200,083, filed Feb. 12, 2021. The entire contents of the above-referenced application are incorporated herein by reference.

### STATEMENT REGARDING FEDERALLY SPONSORED RESEARCH OR DEVELOPMENT

Not Applicable.

### BACKGROUND

The field of medical diagnostics utilizes many different forms of assay technologies. When a patient is suspected of being infected with a microorganism (such as, but not limited to, a bacteria or virus), an assay may be performed on a biological sample from the patient to detect antigens from the microorganism or antibodies directed to the microorganism that are being produced by the patient's immune system.

COVID-19 (coronavirus disease 2019) is the illness resulting from infection with SARS-COV-2 (severe acute respiratory syndrome coronavirus 2) virus. The virus spreads readily from person to person primarily through infected secretions, such as saliva and respiratory droplets or aerosols. Evidence supports spread by both symptomatic and asymptomatic individuals. The virus incubation period ranges from 2-14 days following exposure, with most cases showing symptoms within approximately 5 days after exposure.

SARS-COV-2 nucleic acid amplification testing, such as reverse transcription polymerase chain reaction (RT-PCR), is considered the gold standard for diagnostic testing for current infection, typically using an upper respiratory tract specimen. RT-PCR detects the genetic material of the virus, while antigen tests detect a viral protein (e.g. nucleocapsid). Immunoassays that detect the SARS-COV-2 nucleocapsid antigen are also used for the diagnosis of current infection. Due to the highly contagious nature of this virus and the mode of transmission, the need for additional, less complex testing solutions has been recognized. Antigen testing, as part of a SARS-COV-2 testing strategy, can be used to identify symptomatic and asymptomatic individuals currently infected with SARS-COV-2.

Antigen tests for SARS-COV-2 are particularly desirable during the COVID-19 pandemic, as their use can expand access to faster screening of at-risk individuals. They are useful in responding to suspected outbreaks of COVID-19, particularly where PCR is not immediately available, and they allow for earlier implementation of infection control measures. SARS-COV-2 antigen tests also support outbreak investigations in closed or semi-closed groups and to monitor trends in disease incidence in communities. Where there is widespread community transmission, antigen tests allow for early detection and isolation of positive cases as well as enable effective infection control and contract tracing.

Just as there is a need for new and improved assays for various viruses and viral infections, such as (but not limited to) COVID-19, there is a corresponding need for new and improved reagents and sample handling methods that are utilized therewith. In particular, there is a need for methods and reagents utilized during sample handling for viral inactivation, thereby rendering samples non-infectious and providing safer sample handling conditions. It is to such reagents, kits containing same, and methods of production and use thereof, that the present disclosure is directed.

### BRIEF DESCRIPTION OF THE DRAWINGS

FIG. 1 schematically depicts viral antigen sample flow for two non-limiting examples of laboratory-based viral antigen assay formats, including rt-PCR and high throughput assays. VTM: Viral transfer Media; UTM: Universal transfer media. Throughput ADVIA Centaur 240 tests/hour; Atellica IM 440 test/hour.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, kits, devices, and/or methods disclosed herein can be made and executed without undue experimentation in light of the present disclosure. While the compositions, kits, devices, and/or methods have been described in terms of particular embodiments, it will be apparent to those of skill in the art that variations may be applied to the compositions, kits, devices, and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “sample” as used herein will be understood to include any type of biological sample that may be utilized in accordance with the present disclosure. Examples of fluidic biological samples that may be utilized include, but are not limited to, nasal, nasopharyngeal, anterior nasal, saliva, mucus, sputum, cerebrospinal fluid (CSF), skin, intestinal fluid, intraperitoneal fluid, cystic fluid, whole blood or any portion thereof (i.e., plasma or serum), urine, sweat, interstitial fluid, extracellular fluid, tears, bladder wash, semen, fecal, pleural fluid, combinations thereof, and the like.

Certain non-limiting embodiments of the present disclosure are directed to a viral sample lysis reagent composition effective in inactivating one or more viruses in biological samples prior to performing assays for the detection of viral antigens in the biological sample. The reagent composition comprises a nonionic, octylphenol ethoxylate surfactant and water. The reagent composition may optionally include other components, such as (but not limited to) an antimicrobial agent (such as, but not limited to, an azide or the like), a preservative, or other agent commonly found in a lysis reagent. Alternatively, the sample lysis reagent composition may contain only the surfactant and water.

The virus(es) to be inactivated in the biological sample may be the virus to be assayed and/or any other virus present in the sample. For example (but not by way of limitation), the virus may be a coronavirus, such as (but not limited to) a SARS-COV-2 virus; an influenza virus; a respiratory syncytial virus (RSV); a hepatitis virus; human papillomavirus (HPV); varicella-zoster virus; herpes simplex virus; Epstein-Barr virus; cytomegalovirus (CMV); rotavirus; human immunodeficiency virus (HIV); morbillivirus (measles virus); paramyxovirus (mumps virus); Ebola virus; poliovirus; norovirus; BK virus; West Nile Virus; Zika virus; Dengue virus; a picornavirus; an enterovirus; a parainfluenza virus; a rubivirus; and the like; as well as any combinations thereof.

Any nonionic, octylphenol ethoxylate surfactant known in the art or otherwise contemplated herein may be utilized in accordance with the present disclosure. Non-limiting examples of octylphenol ethoxylate surfactants that may be utilized include polyethylene glycol octylphenyl ether, and in particular, 2-[4-(2,4,4-trimethylpentan-2-yl)phenoxy]ethanol.

The nonionic surfactant may be present in the sample lysis reagent composition at any initial concentration that allows the reagent composition to function in accordance with the present disclosure (i.e., inactivate the virus(es) and not interfere with the antigen assay). In certain non-limiting embodiments, the surfactant is present in the reagent composition at a concentration independently selected from about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, or higher, or a range formed of two of the above values (i.e., a range of from about 0.1 wt % to about 20 wt %, a range of from about 0.5 wt % to about 15 wt %, a range of from about 1 wt % to about 10 wt %, etc.), or any value that falls within a range of two of the above values (i.e., about 2.7 wt %).

In particular (but non-limiting) embodiments, the nonionic surfactant is present in the sample lysis reagent composition at an initial concentration in a range of from about 1 wt % to about 15 wt %. In a particular (but non-limiting) embodiment, the nonionic surfactant is present in the sample lysis reagent composition at an initial concentration in a range of from about 5 wt % to about 10 wt %. In another particular (but non-limiting) embodiment, the nonionic surfactant is present in the sample lysis reagent composition at an initial concentration of about 10 wt %.

Deionized water may be present in the sample lysis reagent composition at any concentration that allows the reagent composition to function in accordance with the present disclosure. For example (but not by way of limitation), deionized water may be present at a concentration selected from about 50 wt %, about 55 wt %, about 60 wt %, about 65 wt %, about 70 wt %, about 75 wt %, about 80 wt %, about 81 wt %, about 82 wt %, about 83 wt %, about 84 wt %, about 85 wt %, about 86 wt %, about 87 wt %, about 88 wt %, about 89 wt %, about 90 wt %, about 91 wt %, about 92 wt %, about 93 wt %, about 94 wt %, about 95 wt %, about 96 wt %, about 97 wt %, about 98 wt %, about 99 wt %, or higher, or a range formed of two of the above values (i.e., a range of from about 85 wt % to about 99 wt %, a range of from about 90 wt % to about 95 wt %, etc.), or any value that falls within a range of two of the above values.

Similarly, any additional components present in the reagent composition (i.e., one or more antimicrobial agent(s), preservative(s), bulking agent(s), etc.) may each be present at any concentration that allows the reagent compositions formed therefrom to function in accordance with the present disclosure. For example (but not by way of limitation), each additional component may be present at a concentration independently selected from about 0.001 wt %, about 0.002 wt %, about 0.003 wt %, about 0.004 wt %, about 0.005 wt %, about 0.006 wt %, about 0.007 wt %, about 0.008 wt %, about 0.009 wt %, about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 3 wt %, about 4 wt %, about 5 wt %, about 6 wt %, about 7 wt %, about 8 wt %, about 9 wt %, about 10 wt %, about 15 wt %, about 20 wt %, about 25 wt %, or higher, or a range formed of two of the above values (i.e., a range of from about 0.001 wt % to about 10 wt %, a range of from about 0.1 wt % to about 5 wt %, a range of from about 2 wt % to about 60 wt %, etc.), or any value that falls within a range of two of the above values.

The sample lysis reagent composition of the present disclosure is substantially stable at a desired temperature for a desired period of time. For example (but not by way of limitation), the sample lysis reagent composition may be substantially stable for at least about 7 days, at least about 14 days, at least about 28 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 2 years, at least about 3 years or longer. Also, the sample lysis reagent composition may be stored at room temperature (i.e., a range of from about 18° C. to about 25° C.), or stored under refrigerated or frozen conditions. When a preservative is present in the sample lysis reagent composition, the preservative utilized may be selected based upon the particular storage conditions and storage period desired.

The sample lysis reagent compositions of the present disclosure may be provided in any form that allows the reagent composition to function in accordance with the present disclosure. For example, but not by way of limitation, the reagent composition may be provided in liquid form. Alternatively, the reagent composition may be freeze-dried or lyophilized and provided in the form of a dry reagent.

The viral antigen assay may detect any type of virus that is capable of infecting a patient (such as, but not limited to, a human or domestic animal) and causing a disease state, and for which a particular antigen is known. Examples of viruses detected by viral antigen assays in accordance with the present disclosure include, but are not limited to, a coronavirus, such as (but not limited to) a SARS-COV-2 virus; an influenza virus; a respiratory syncytial virus (RSV); a hepatitis virus; human papillomavirus (HPV); varicella-zoster virus; herpes simplex virus; Epstein-Barr virus; cytomegalovirus (CMV); rotavirus; human immunodeficiency virus (HIV); morbillivirus (measles virus); paramyxovirus (mumps virus); Ebola virus; poliovirus; norovirus; BK virus; West Nile Virus; Zika virus; Dengue virus; a picornavirus; an enterovirus; a parainfluenza virus; a rubivirus; and the like; as well as any combinations thereof.

Particular (but non-limiting) examples of viruses detected by viral antigen assays in accordance with the present disclosure include a coronavirus (such as, but not limited to SARS-COV-2 virus) and an influenza virus.

The SARS-COV-2 antigen assays with which the sample lysis reagent compositions of the present disclosure are utilized may detect any known antigen of the virus. Non-limiting examples of antigens that may be detected include at least a portion of the Nucleocapsid (N) protein, at least a portion of the Spike (S) protein, at least a portion of the Matrix (M) protein, at least a portion of the Envelope (E) protein, at least a portion of the ssRNA, combinations thereof, and the like.

Certain non-limiting embodiments of the present disclosure are directed to kits that contain one or more of any of the sample lysis reagent compositions described or otherwise contemplated herein.

The kits may contain one or more other reagents/components for use in sample handling and/or assays in accordance with the present disclosure. For example (but not by way of limitation), the kits may further include at least one reagent composition for use in performing a viral antigen assay (such as, but not limited to, a SARS-COV-2 or other coronavirus antigen assay, or an influenza antigen assay). For example, but not by way of limitation, the kits may further include at least one reagent that specifically binds to the antigen (such as, but not limited to, an antibody that specifically binds to the viral antigen (i.e., an anti-SARS-COV-2 antibody or an anti-influenza antibody, etc.), at least one reagent utilized in the particular assay format utilized, and/or at least one viral antigen-containing reagent, as well as various combinations thereof. When the reagent contains a viral antigen, the viral antigen may be the same or different from the viral antigen being assayed. Non-limiting examples of viral antigen-containing reagents that fall within the scope of the present disclosure include a calibrator containing a viral antigen, a quality control solution containing a viral antigen, and the like.

Non-limiting examples of coronavirus antigen-containing reagents that fall within the scope of the present disclosure include a calibrator containing a coronavirus antigen, a quality control solution containing a coronavirus antigen, and the like. Non-limiting examples of SARS-COV-2 antigen-containing reagents that fall within the scope of the present disclosure include a calibrator containing a SARS-COV-2 antigen, a quality control solution containing a SARS-COV-2 antigen, and the like. Non-limiting examples of influenza antigen-containing reagents that fall within the scope of the present disclosure include a calibrator containing an influenza antigen, a quality control solution containing an influenza antigen, and the like.

Alternatively, and/or in addition thereto, the kits may contain one or more sample collection tubes.

In a particular (but non-limiting) embodiment, the kits may contain one or more sample collection tubes that have any of the sample lysis reagent compositions described or otherwise contemplated herein associated therewith.

The sample lysis reagent compositions present in any of the kits described herein may be provided in any form that allows them to function in accordance with the present disclosure. For example, but not by way of limitation, each of the reagents may be provided in liquid form and disposed in bulk and/or single aliquot form within the kit. Alternatively, in a particular (but non-limiting) embodiment, one or more of the reagents may be disposed in the kit in the form of a single aliquot lyophilized reagent. The use of dried reagents in kits and microfluidics devices is described in detail in U.S. Pat. No. 9,244,085 (Samproni), the entire contents of which are hereby expressly incorporated herein by reference. When one or more of the reagents are lyophilized, the kit may further contain an excipient for reconstitution of the reagent(s); alternatively, the sample itself may function as an excipient when mixed with the lyophilized reagent.

When the kit contains a sample collection tube in which the sample lysis reagent composition is disposed, and when the sample lysis reagent is in liquid form, the sample lysis reagent composition may be disposed in the sample collection tube at any volume that allows the sample lysis reagent composition to function in accordance with the present disclosure. Non-limiting examples of volumes per mL volume of the sample collection tube that may be utilized in accordance with the present disclosure include about 1 μL, about 2 μL, about 3 μL, about 4 μL, about 5 μL, about 6 μL, about 7 μL, about 8 μL, about 9 μL, about 10 μL, about 11 μL, about 12 μL, about 13 μL, about 14 μL, about 15 μL, about 16 μL, about 17 μL, about 18 μL, about 19 μL, about 20 μL, about 21 μL, about 22 μL, about 23 μL, about 24 μL, about 25 μL, about 26 μL, about 27 μL, about 28 μL, about 29 μL, about 30 μL, about 31 μL, about 32 μL, about 33 μL, about 34 μL, about 35 μL, about 36 μL, about 37 μL, about 38 μL, about 39 μL, about 40 μL, about 41 μL, about 42 μL, about 43 μL, about 44 μL, about 45 μL, about 46 μL, about 47 μL, about 48 μL, about 49 μL, about 50 μL, about 51 μL, about 52 μL, about 53 μL, about 54 μL, about 55 μL, about 56 μL, about 57 μL, about 58 μL, about 59 μL, about 60 μL, about 61 μL, about 62 μL, about 63 μL, about 64 μL, about 65 μL, about 66 μL, about 67 μL, about 68 μL, about 69 μL, about 70 μL, about 71 μL, about 72 μL, about 73 μL, about 74 μL, about 75 μL, about 76 μL, about 77 μL, about 78 μL, about 79 μL, about 80 μL, about 81 μL, about 82 μL, about 83 μL, about 84 μL, about 85 μL, about 86 μL, about 87 μL, about 88 μL, about 89 μL, about 90 μL, about 91 μL, about 92 μL, about 93 μL, about 94 μL, about 95 μL, about 96 μL, about 97 μL, about 98 μL, about 99 μL, about 100 μL, about 101 μL, about 102 μL, about 103 μL, about 104 μL, about 105 μL, about 106 μL, about 107 μL, about 108 μL, about 109 μL, about 110 μL, about 115 μL, about 120 μL, about 125 μL, about 130 μL, about 135 μL, about 140 μL, about 145 μL, about 150 μL, or higher, or a range formed of two of the above values (i.e., a range of from about 1 μL to about 150 μL, a range of from about 10 μL to about 100 μL, etc.), or any value that falls within a range of two of the above values.

In particular (but non-limiting) embodiments, the sample lysis reagent composition is disposed in the sample collection tube in an amount in a range of from about 10 μL to about 100 μL per mL volume of the sample collection tube. In a particular (but non-limiting) embodiment, the sample lysis reagent composition is disposed in the sample collection tube in an amount of about 50 μL per mL volume of the sample collection tube.

Alternatively, when the kit contains a sample collection tube in which the sample lysis reagent composition is disposed, the sample lysis reagent composition may be disposed in the sample collection tube in a freeze-dried or lyophilized form. In this manner, the sample acts as an excipient(s) for dissolution of the lyophilized reagent; alternatively, the kit may include a separate excipient which is disposed in the sample collection tube for reconstitution of the sample lysis reagent composition prior to addition of the sample to the sample collection tube. In addition, in certain particular (but non-limiting) embodiments, the use of a sample lysis reagent composition in lyophilized form would allow the lyophilized reagent to be attached to a sidewall of the sample collection tube.

In addition to the assay components/reagents described in detail herein above, any of the kits described or otherwise contemplated herein may further contain other reagent(s) for conducting any of the particular assays described or otherwise contemplated herein. The nature of these additional reagent(s) will depend upon the particular assay format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the cross-reactivity and stability of the components/reagents. In addition, the kit may include a microfluidics device in which the components/reagents are disposed.

The relative amounts of the various components/reagents in the kits can vary widely to provide for concentrations of the components/reagents that substantially optimize the reactions that need to occur during the assay methods and further to optimize substantially the sensitivity of an assay. Under appropriate circumstances, one or more of the components/reagents in the kit can be provided as a dry powder, such as a lyophilized powder, and the kit may further include excipient(s) for dissolution of the dried reagents; in this manner, a reagent solution having the appropriate concentrations for performing a method or assay in accordance with the present disclosure can be obtained from these components. Non-limiting examples of other reagents that can be included in the kits include wash solutions, dilution solutions, excipients, interference solutions, positive controls, negative controls, and the like. In addition, the kit can further include a set of written instructions explaining how to use the kit. A kit of this nature can be used in any of the methods described or otherwise contemplated herein.

Certain non-limiting embodiments of the present disclosure are directed to a method of producing any of the sample lysis reagent compositions for a SARS-COV-2 antigen assay, as described or otherwise contemplated herein. In the method, water is added to a desired amount of the nonionic surfactant until a desired surfactant concentration (as described in detail herein above) is achieved. Then the solution is mixed well and stored at a desired temperature for a desired period (as described herein).

In addition, the method of producing any of the sample lysis reagent compositions may contain one or more additional steps in order to form the reagent compositions described herein.

Certain non-limiting embodiments of the present disclosure are directed to a method of inactivating at least one virus present in a biological sample. In the method, any of the sample lysis reagent compositions described or otherwise contemplated herein is added to a biological sample to form a mixture, and the mixture is incubated for a desired period of time and at a temperature sufficient to substantially inactivate the at least one virus.

Any biological samples that potentially contain evidence of infection with one or more viruses may be utilized in accordance with the present disclosure. For example, but not by way of limitation, the biological sample may be nasal, nasopharyngeal, anterior nasal, saliva, mucus, sputum, cerebrospinal fluid, intestinal fluid, intraperitoneal fluid, cystic fluid, whole blood or any portion thereof, urine, sweat, interstitial fluid, extracellular fluid, tears, bladder wash, semen, fecal, pleural fluid, and combinations thereof.

In a particular (but non-limiting) embodiment, the biological sample is selected from the group consisting of nasal, nasopharyngeal, anterior nasal, saliva, mucus, sputum, and combinations thereof.

The sample lysis reagent compositions may be utilized to inactivate any of the viruses described or otherwise contemplated herein. In particular (but non-limiting) embodiments, the one or more viruses to be inactivated includes a coronavirus, such as (but not limited to) a SARS-COV-2 virus; an influenza virus; a respiratory syncytial virus (RSV); a hepatitis virus; human papillomavirus (HPV); varicella-zoster virus; herpes simplex virus; Epstein-Barr virus; cytomegalovirus (CMV); rotavirus; human immunodeficiency virus (HIV); morbillivirus (measles virus); paramyxovirus (mumps virus); Ebola virus; poliovirus; norovirus; BK virus; West Nile Virus; Zika virus; Dengue virus; a picornavirus; an enterovirus; a parainfluenza virus; a rubivirus; and the like; as well as any combinations thereof.

The sample lysis reagent composition may be added to the biological sample at any volume (and thus at any final concentration, based upon the initial concentration of the sample lysis reagent composition, as described in detail herein above) that allows the sample lysis reagent composition to function in accordance with the present disclosure. Non-limiting examples of volumes per mL of biological sample that may be utilized in accordance with the present disclosure include about 1 μL, about 2 μL, about 3 μL, about 4 μL, about 5 μL, about 6 μL, about 7 μL, about 8 μL, about 9 μL, about 10 μL, about 11 μL, about 12 μL, about 13 μL, about 14 μL, about 15 μL, about 16 μL, about 17 μL, about 18 μL, about 19 μL, about 20 μL, about 21 μL, about 22 μL, about 23 μL, about 24 μL, about 25 μL, about 26 μL, about 27 μL, about 28 μL, about 29 μL, about 30 μL, about 31 μL, about 32 μL, about 33 μL, about 34 μL, about 35 μL, about 36 μL, about 37 μL, about 38 μL, about 39 μL, about 40 μL, about 41 μL, about 42 μL, about 43 μL, about 44 μL, about 45 μL, about 46 μL, about 47 μL, about 48 μL, about 49 μL, about 50 μL, about 51 μL, about 52 μL, about 53 μL, about 54 μL, about 55 μL, about 56 μL, about 57 μL, about 58 μL, about 59 μL, about 60 μL, about 61 μL, about 62 μL, about 63 μL, about 64 μL, about 65 μL, about 66 μL, about 67 μL, about 68 μL, about 69 μL, about 70 μL, about 71 μL, about 72 μL, about 73 μL, about 74 μL, about 75 μL, about 76 μL, about 77 μL, about 78 μL, about 79 μL, about 80 μL, about 81 μL, about 82 μL, about 83 μL, about 84 μL, about 85 μL, about 86 μL, about 87 μL, about 88 μL, about 89 μL, about 90 μL, about 91 μL, about 92 μL, about 93 μL, about 94 μL, about 95 μL, about 96 μL, about 97 μL, about 98 μL, about 99 μL, about 100 μL, about 101 μL, about 102 μL, about 103 μL, about 104 μL, about 105 μL, about 106 μL, about 107 μL, about 108 μL, about 109 μL, about 110 μL, about 115 μL, about 120 μL, about 125 μL, about 130 μL, about 135 μL, about 140 μL, about 145 μL, about 150 μL, or higher, or a range formed of two of the above values (i.e., a range of from about 1 μL to about 150 μL, a range of from about 10 μL to about 100 μL, etc.), or any value that falls within a range of two of the above values.

In particular (but non-limiting) embodiments, the sample lysis reagent composition is added to the biological sample in an amount in a range of from about 10 μL to about 100 μL per ml of biological sample present. In a particular (but non-limiting) embodiment, the sample lysis reagent composition is added to the biological sample in an amount of about 50 μL per mL of biological sample.

The sample lysis reagent composition may be present in the sample lysis reagent composition/biological sample mixture at any final concentration that allows the sample lysis reagent composition to function in accordance with the present disclosure (i.e., inactivate the virus(es) and not interfere with the antigen assay). In certain non-limiting embodiments, the surfactant is present in the reagent composition at a concentration independently selected from about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 3 wt %, about 4 wt %, about 5 wt %, or higher, or a range formed of two of the above values (i.e., a range of from about 0.01 wt % to about 2 wt %, a range of from about 0.01 wt % to about 1 wt %, a range of from about 0.05 wt % to about 1 wt %, etc.), or any value that falls within a range of two of the above values.

As stated herein above, the mixture is incubated for any desired period of time and at any temperature sufficient to substantially inactivate the virus(es). For example (but not by way of limitation), the mixture may be incubated at a temperature of about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., or higher, as well as any range formed of two of the above values. In a particular (but non-limiting) embodiment, the mixture is incubated at room temperature (i.e., a temperature in a range of from about 18° C. to about 25° C.).

Also, for example (but not by way of limitation), the mixture may be incubated for at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 11 minutes, at least about 12 minutes, at least about 13 minutes, at least about 14 minutes, at least about 15 minutes, at least about 16 minutes, at least about 17 minutes, at least about 18 minutes, at least about 19 minutes, at least about 20 minutes, at least about 21 minutes, at least about 22 minutes, at least about 23 minutes, at least about 24 minutes, at least about 25 minutes, at least about 26 minutes, at least about 27 minutes, at least about 28 minutes, at least about 29 minutes, at least about 30 minutes, at least about 45 minutes, at least about 60 minutes, at least about 90 minutes, at least about 120 minutes, or longer, as well as any range formed of two of the above values (i.e., a range of from about 5 minutes to about 20 minutes, etc.). In a particular (but non-limiting) embodiment, the mixture is incubated for about 10 minutes.

One or more of the steps of the methods of the present disclosure may be performed simultaneously or wholly or partially sequentially with one or more assay steps for detection of one or more viral antigens in the biological sample.

In addition, the mixture containing inactivated virus may be stored for any desired period of time and at any temperature, so long as the antigens present in the biological sample can be detected. Particular (but non-limiting) examples of storage conditions that can be utilized include up to about 4 hours at room temperature, up to about 3 days at 2-8° C., and longer when stored at ≤−20° C.

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are simply meant to be exemplary, not exhaustive.

### Example 1

This Example describes the preparation of a sample lysis reagent composition constructed in accordance with the present disclosure.

A required amount of Triton X-100 (2-[4-(2,4,4-trimethylpentan-2-yl)phenoxy]ethanol) was disposed in a container, and then deionized water was added until a Triton X-100 concentration of about 10 wt % was obtained. The solution was then mixed to ensure adequate distribution of the Triton X-100 therein. For example (but not by way of limitation), the Triton X-100 and deionized water solution was mixed for 120-240 minutes on a roller mixer set at 30-45 or a 3D Orbital Mixer set at 70-90 RPM to ensure proper mixing.

The pH of the solution was then measured at room temperature (i.e., in a range of from about 18° C. to about 25° C.) to verify that the pH was in a range of from about 4.0 to about 6.0. The solution was also optionally filtered through a 0.2 μm filter, if desired.

The resulting sample lysis reagent composition was then stored at room temperature or in a range of from about 2° C. to about 8° C.

### Example 2

This Example discloses an infectivity determination for sample lysis reagent composition treatment of SARS-COV-2 culture fluid. This study was performed to determine the extent to which Triton X-100 (2-[4-(2,4,4-trimethylpentan-2-yl)phenoxy]ethanol) has the ability to inactivate SARS-COV-2 without destroying the ability of the viral proteins to be detected in an antigen-based detection assay.

### Materials and Methods:

The sample lysis reagent composition produced as in Example 1 but with a Triton X-100 concentration of about 0.5 wt % was utilized to test the efficacy to inactivate SARS-COV-2 (Isolate: USAWA1/2020). The lower initial Triton X-100 concentration was utilized to ensure that the cells being tested remain intact when exposed to the sample lysis reagent composition; however, retaining intact cells is not a requirement for the SARS-COV-2 antigen assays, and thus higher initial surfactant concentrations can be employed in the sample lysis reagent compositions utilized in accordance with the methods of the present disclosure.

SARS-COV-2-infected VeroE6 cell culture fluid (ZeptoMetrix, Buffalo, NY Part #0610587CF Lot #544347) was grown and frozen (−65° C. or below) for experimental procedures. The culture fluid was thawed, diluted to 2×10⁶ TCID₅₀/mL, and combined with the lysis buffer at a 1:10 dilution. The lysis buffer and the virus had a contact time of 1, 2, 5, and 10 minutes before infectivity plates were set up. As a control, the buffers were combined with uninfected VeroE6 cell culture fluid for a contact time of 10 minutes at a 1:10 dilution to observe if cell toxicity would affect the infectivity test determination. All samples were tested in quadruplicate and observed for 7 days via microscopic visualization of cytopathic effects (CPE).

On Day 0, SARS-COV-2 was diluted to 2×10⁶ TCID₅₀/mL and combined with 0.5% Triton X-100 at a 1:10 dilution. The mixtures were incubated for 1, 2, 5, and 10 minutes at room temperature. After incubations, the mixtures were transferred to 96-well plates. Control wells that contained only cells did not have any lysis buffer added. For the negative controls, 0.5% Triton was mixed with 2% MEM media at a 1:10 dilution for 10 minutes. After the incubation, the cells/reagent mixture was transferred to the 96-well plate. Control wells that contained only cells did not have any lysis reagent added. For the positive control, a TCID₅₀ assay was performed using the VeroE6 cell culture fluid. All cultures were incubated at 37° C. for 7 days. VeroE6 cells were added to the wells after all conditions were set up. All conditions in the 96-well plates were observed on 3- and 7-days post-infection (dpi) and recorded on plate maps. Cytopathic effects (CPE) were scored as shown in Table 1.

TABLE 1
CPE Scoring System

| SCORE | CPE |
|---|---|
| − | Absence of CPE (Healthy cells, Negative CPE) |
| + | Presence of SARS-CoV-2 CPE (subtotal destruction) |
| ++ | Presence of SARS-CoV-2 CPE (total destruction) |
| +/− | Presence of cytotoxicity or Undetermined CPE |

### Results:

The infectious SARS-COV-2 virus positive controls demonstrated visible CPE in the TCID₅₀ Assay, resulting in total destruction by 7 days post-infection in wells 1-7. This indicates that viral infection of the VeroE6 cells occurred properly in the absence of any lysis reagent. The positive control had titer at a value of 1.05×10⁶ TCID₅₀/mL. Each negative control consisting of VeroE6 cells-only remained healthy in each plate over the course of the assay.

Uninfected VeroE6 cells were also exposed to 0.5% Triton at a 1:10 dilution as a control for cytotoxicity. VeroE6 cells treated with 0.5% Triton at a 1:10 ratio didn't show signs of cytotoxicity after days 3 and 7 post-treatment, making this ratio effective to treat SARS-CoV-2 and not interfere with overall cellular health.

SARS-COV-2 infection can cause total or subtotal destruction of the VeroE6 cell monolayer. Upon infection, many of the cells rapidly shrink, become dense, and detach from the monolayer. This phenomenon was observed in the VeroE6 cells infected with SARS-COV-2. SARS-COV-2 treated with 0.5% Triton at a 1:10 ratio for 1, 2, and 5 minutes demonstrated cytopathic effects during the course of the assay. SARS-COV-2 incubated for 1 minute had the highest number of wells show CPE. The effect decreased as the incubation time increased. SARS-COV-2 treated with 0.5% Triton at a 1:10 dilution was not completely effective at inactivating SARS-COV-2 virus when exposed for 1, 2, and 5 minutes. (Table 2).

Infectious SARS-COV-2 treated with 0.5% Triton at a 1:10 dilution for 10 minutes didn't show any visible CPE, indicating that 0.5% Triton added at a 1:10 dilution is effective at inactivating SARS-COV-2 virus when exposed for 10 minutes or more (Table 2).

TABLE 2
Sample Results for SARS-CoV-2 +
0.5% Triton X-100 at 1:10 Dilution

| | |
|---|---|
| Exposure Time | Result |

| | | |
|---|---|---|
| 1 | minute | 1.86 × 10⁴ TCID₅₀/mL |
| 2 | minutes | 5.25 × 103 TCID₅₀/mL |
| 5 | minutes | 2 positive wells |
| 10 | minutes | Negative |

The acceptance criteria were as follows: (1) the positive control infectious SARS-CoV-2 culture fluid must show CPE. (2) All cell-only control wells must remain healthy throughout the 7-day assay. (3) The 0.5% Triton reagent effects on healthy cells must allow for reading of the infectivity plates (i.e., cell death must not interfere with CPE).

### Conclusions:

The cell-only Negative Controls remained healthy over the course of the assay in all testing plates. The positive control SARS-COV-2 culture fluids demonstrated infection CPE over the course of the assay. Therefore, the acceptance criteria of the controls were met. The VeroE6 cell-only wells did not show any signs of CPE. SARS-COV-2 treated with 0.5% Triton at a 1:10 dilution is not completely effective at inactivating SARS-COV-2 virus when exposed for 1, 2, and 5 minutes. SARS-COV-2 treated with 0.5% Triton at a 1:10 dilution was effective at inactivating SARS-COV-2 virus when exposed for 10 minutes or more.

### Example 3

This Example provides one non-limiting protocol for a method of using the sample lysis reagent composition to inactivate virus in a biological sample.

A nasopharyngeal (NP) or anterior nasal (AN) swab sample tube was allowed to thaw, if necessary. The NP/AP swab sample tube was then vortexed for 10 seconds. The cap was carefully removed from the sample tube, and the swab was rolled at least 3 times against the side of the tube and then removed. The swab was subsequently disposed of into a biohazardous waste collection container.

CoV2Ag sample lysis reagent (prepared as described above in Example 1) was then dispensed into the NP/AP swab sample tube at a quantity of 50 μL (or 2 drops) per ml of sample; for example, this required 150 μL (or 6 drops) of CoV2Ag Sample Lysis Reagent for a 3 ml sample (or 5 μL sample lysis reagent for a 100 μL sample). This mixture was then mixed gently.

The mixture was incubated for 10 minutes at room temperature to inactivate the sample. The inactivated sample was then optionally transferred to an appropriate sample cup or other assay device to be placed on the assay system (or for performing a manual assay).

The inactivated sample can be tested as soon as the incubation step is complete. Alternatively, the inactivated samples can be stored at room temperature for a short period of time or frozen for a longer period of time prior to performing the assay. For example (but not by way of limitation), inactivated samples are stable for up to 4 hours onboard the system, and inactivated samples can be stored at 2-8° C. for up to 3 days, or at ≤−20° C. for a longer storage period. The inactivated samples should not be stored in a frost-free freezer, and more than 2 freeze-thaw cycles should be avoided.

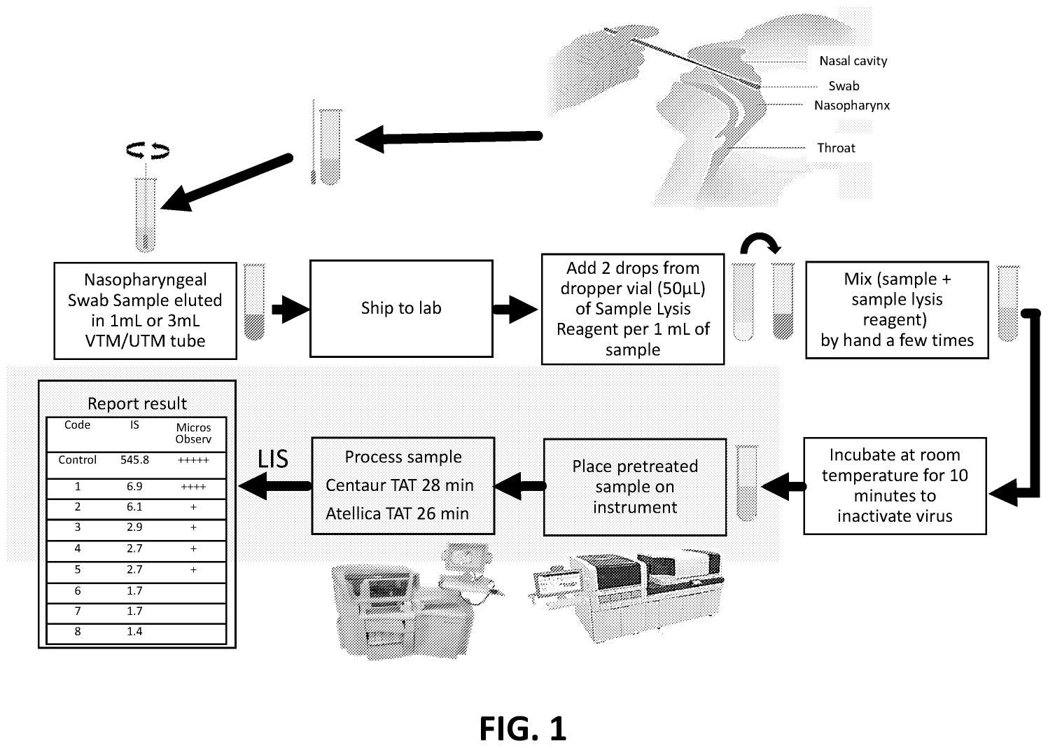

FIG. 1 provides a schematic illustrating sample flow through two types of laboratory-based assays (rt-PCR and high throughput SARS-COV-2 antigen assays), and includes the steps of the methods of the present disclosure (i.e., addition of sample lysis reagent composition to sample, mixing, and incubation to inactivate virus). However, this schematic is for purposes of example only and non-limiting of the present disclosure; as described herein, it will be understood that different samples, different assays, and different sample flow steps may be utilized. Further, the particular sample lysis reagent composition concentrations and incubation conditions listed in FIG. 1 are also for purposes of example only and non-limiting of the methods disclosed or otherwise contemplated herein.

Thus, in accordance with the present disclosure, there have been provided compositions, kits, and devices, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method of inactivating at least one virus present in a biological sample, the method comprising the steps of:
(i) adding a sample lysis reagent composition to the biological sample to form a mixture, wherein the sample lysis reagent composition comprises a nonionic, octylphenol ethoxylate surfactant and water, and wherein the surfactant is present in the sample lysis reagent composition at an initial concentration in a range of from about 1 wt % to about 15 wt %; and
(ii) incubating the mixture for a period in a range of from about 5 minutes to about 20 minutes and at a temperature sufficient to substantially inactivate the at least one virus.

2. The method of claim 1, wherein the nonionic surfactant comprises polyethylene glycol octylphenyl ether.

3. The method of claim 1, wherein the nonionic surfactant comprises 2-[4-(2,4,4-trimethylpentan-2-yl)phenoxy]ethanol.

4. The method of claim 1, wherein the surfactant is present in the sample lysis reagent composition at an initial concentration in a range of from about 5 wt % to about 10 wt %.

5. The method of claim 1, wherein the surfactant is present in the sample lysis reagent composition at an initial concentration of about 10 wt %.

6. The method of claim 1, wherein the sample lysis reagent composition is added to the biological sample at a concentration in range of from about 10 μL to about 100 μL per mL of biological sample.

7. The method of claim 6, wherein the sample lysis reagent composition is added to the biological sample at a concentration of about 50 μL per ml of biological sample.

8. The method of claim 1, wherein the surfactant is present in the mixture at a final concentration in a range of from about 0.01 wt % to about 2 wt %.

9. The method of claim 1, wherein the mixture is incubated at a temperature in a range of from about 18° C. to about 25° C.

10. The method of claim 1, wherein the mixture is incubated for about 10 minutes.

11. The method of claim 1, wherein the biological sample is selected from the group consisting of nasal, nasopharyngeal, anterior nasal, saliva, mucus, sputum, cerebrospinal fluid, intestinal fluid, intraperitoneal fluid, cystic fluid, whole blood or any portion thereof, urine, sweat, interstitial fluid, extracellular fluid, tears, bladder wash, semen, fecal, pleural fluid, and combinations thereof.

12. The method of claim 1, wherein the biological sample is selected from the group consisting of nasal, nasopharyngeal, anterior nasal, saliva, mucus, sputum, and combinations thereof.

13. The method of claim 1, further comprising the step of:
(iii) storing the incubated mixture at a temperature in a range of from about −80° C. to about 8° C.

14. The method of claim 1, wherein the virus is a coronavirus.

15. The method of claim 14, wherein the coronavirus is severe acute respiratory syndrome coronavirus 2 (SARS-COV-2).

16. The method of claim 1, wherein the virus is an influenza virus.

17. A kit, comprising:
at least one sample lysis reagent composition comprising a nonionic, octylphenol ethoxylate surfactant and water, wherein the surfactant is present in the composition in a range of from about 1 wt % to about 15 wt %; and
at least one reagent for use in performing a viral antigen assay.

18. The kit of claim 17, wherein the nonionic surfactant comprises polyethylene glycol octylphenyl ether.

19. The kit of claim 17, wherein the surfactant comprises 2-[4-(2,4,4-trimethylpentan-2-yl)phenoxy]ethanol.

20. The kit of claim 17, wherein the surfactant is present in the sample lysis reagent composition at a concentration in a range of from about 5 wt % to about 10 wt %.

21. The kit of claim 17, wherein the surfactant is present in the sample lysis reagent composition at a concentration of about 10 wt %.

22. The kit of claim 17, wherein the at least one reagent for use in performing a viral antigen assay is selected from the group consisting of an antibody that specifically binds to a viral antigen, a calibrator containing a viral antigen, a quality control solution containing a viral antigen, and combinations thereof.

23. The kit of claim 17, wherein the viral antigen is from a coronavirus.

24. The kit of claim 23, wherein the coronavirus is severe acute respiratory syndrome coronavirus 2 (SARS-COV-2).

25. The kit of claim 17, wherein the viral antigen is from an influenza virus.

26. A kit, comprising:
at least one sample collection tube having at least one sample lysis reagent composition associated therewith, wherein the sample lysis reagent composition comprises a nonionic, octylphenol ethoxylate surfactant and water, wherein the surfactant is present in a range of from about 1 wt % to about 15 wt %.

27. The kit of claim 26, wherein the nonionic surfactant comprises polyethylene glycol octylphenyl ether.

28. The kit of claim 26, wherein the surfactant comprises 2-[4-(2,4,4-trimethylpentan-2-yl)phenoxy]ethanol.

29. The kit of claim 26, wherein the surfactant is present in the sample lysis reagent composition at a concentration in a range of from about 5 wt % to about 10 wt %.

30. The kit of claim 26, wherein the surfactant is present in the sample lysis reagent composition at a concentration of about 10 wt %.

31. The kit of claim 26, wherein an amount of sample lysis reagent composition present in the sample collection tube is in a range of from about 10 μL to about 100 μL per mL volume of the sample collection tube.

32. The kit of claim 26, wherein an amount of sample lysis reagent composition present in the sample collection tube at a concentration of about 50 μL per ml of a volume of the sample collection tube.

33. The kit of claim 26, wherein the nonionic, octylphenol ethoxylate surfactant is lyophilized.

34. The kit of claim 33, wherein the nonionic, octylphenol ethoxylate surfactant is attached to a sidewall of the sample collection tube.
